SEQUENCE LISTING (8 sequences)

SEQ ID NO: 1 - protein, 735 aa, Adeno-associated dependoparvovirus A [sequence omitted]

SEQ ID NO: 2 - protein, 735 aa, Adeno-associated dependoparvovirus A
MAADGYLPDWLEDTLSEGIRQWWKLKPGPPPPKPAERPKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEVQYTSNYAKSANVDFTVDNNGLYTEPRPIGTRYLTRPL

SEQ ID NO: 3 - DNA, 2208 bp, Adeno-associated dependoparvovirus A
  source 1..2208: mol_type = genomic DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 4 - DNA, 2208 bp, Adeno-associated dependoparvovirus A
  source 1..2208: mol_type = genomic DNA
atggctgccgatggttatcttccagattggctcgaggacactctctctgaaggaataagacagtggtggaagctcaaacctggcccaccaccaccaaagcccgcagagcggcctaaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccccagattctcatcaagaacaccccggtacctgcgaatccttcgaccaccttcagtgcggcaaagtttgcttccttcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 5 - DNA, 7322 bp, Adeno-associated dependoparvovirus A
  source 1..7322: mol_type = genomic DNA
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtaccttcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggccaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaattacgtgttaatcaataaaccggttaattcgtgtcagttgaactttggtctcatgtcgttattatcttatctggtcaccagatacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtcacgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggtaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataagcatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaa

SEQ ID NO: 6 - DNA, 7325 bp, Adeno-associated dependoparvovirus A
  source 1..7325: mol_type = genomic DNA
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcctaaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccccagattctcatcaagaacaccccggtacctgcgaatccttcgaccaccttcagtgcggcaaagtttgcttccttcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattacgtgttaatcaataaaccggttaattcgtgtcagttgaactttggtctcatgtcgttattatcttatctggtcaccagatacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtcacgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggtaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataagcatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaa

SEQ ID NO: 7 - DNA, 22 bp, synthetic construct
  source 1..22: mol_type = other DNA
gcggaagcttcgatcaactacg

SEQ ID NO: 8 - DNA, 23 bp, Adeno-associated dependoparvovirus A
  source 1..23: mol_type = other DNA
cgcagagaccaaagttcaactga

(19) United States
(12) Patent Application Publication
(10) Pub. No.: US 2026/0124323 A1
(43) Pub. Date: May 7, 2026

(54) ADENO-ASSOCIATED VIRUS VARIANT AND USE THEREOF

(71) Applicant: UIF (UNIVERSITY INDUSTRY FOUNDATION), YONSEI UNIVERSITY, Seoul (KR)

(72) Inventors: Jae-Hyung JANG, Seoul (KR); Junwon LEE, Seoul (KR); Yoojin KIM, Seoul (KR); Han JEONG, Seoul (KR)

(73) Assignee: UIF (UNIVERSITY INDUSTRY FOUNDATION), YONSEI UNIVERSITY, Seoul (KR)

(21) Appl. No.: 19/375,466

(22) Filed: Oct. 31, 2025

(30) Foreign Application Priority Data
Nov. 1, 2024 (KR) 10-2024-0153681

(51) Int. Cl. A61K 48/00; C07K 14/005; C12N 15/86

(52) U.S. Cl. CPC A61K 48/0058; A61K 48/0066; C07K 14/005; C12N 15/86; C12N 2750/14122; C12N 2750/14143

(57) ABSTRACT

Disclosed are adeno-associated virus (AAV) capsid protein variants that have improved infectivity and transduction efficiency for target cells, and uses thereof. Specifically, the disclosure relates to AAV2 variant capsid proteins selected by directed evolution for gene delivery to retinal cells, recombinant AAV vectors comprising the same, and uses thereof as gene delivery vehicles to retinal cells.

### CROSS-REFERENCE TO RELATED APPLICATION

This application is based on and claims priority under 35 U.S.C. § 119 to Korean Patent Application No. 10-2024-0153681, filed on Nov. 1, 2024, in the Korean Intellectual Property Office, the disclosure of which is incorporated by reference herein in its entirety.

### INCORPORATION BY REFERENCE OF SEQUENCE LISTING

The instant application contains a Sequence Listing which has been filed electronically in xml format and is hereby incorporated by reference in its entirety. Said xml copy, created on Oct. 30, 2025, is named Q315057_Sequence_listing.xml and is 28,456 bytes in size.

### BACKGROUND

### 1. Field

The disclosure relates to adeno-associated virus (AAV) capsid protein variants having improved infectivity and transduction efficiency for target cells, and uses thereof. More specifically, the disclosure relates to AAV2 variant capsid proteins selected by directed evolution for gene delivery to retinal cells, recombinant AAV vectors comprising the same, and uses thereof as gene delivery vehicles to retinal cells.

This study was supported by Samsung Science & Technology Foundation (Project Number: SRFC-MA2202-08).

### 2. Description of the Related Art

Gene therapy is a method of treating genetic disorders by introducing normal genes into human cells to correct or compensate for defective or abnormal genes, and has become a key approach for treating various genetic disorders. Adeno-associated viruses (AAVs) are widely used gene delivery vectors in gene therapy because they can infect a wide variety of tissues, are nonpathogenic, and exhibit low immunogenicity.

AAVs exist in various serotypes, and it is known that the host and viral properties differ depending on the serotype. For example, AAV serotype 2 (AAV2) can infect a wide range of cells, whereas AAV serotype 1 (AAV1), AAV serotype 5 (AAV5), and AAV serotype 6 (AAV6) exhibit tissue-specific infection patterns compared to AAV2; with AAV1 efficiently delivers genes to muscle, liver, airway, and the central nervous system; AAV5 to the central nervous system, liver, and retina; and AAV6 to heart, muscle, and liver.

However, to be used as gene delivery vectors for gene therapy, AAV vectors must possess enhanced tissue specificity and improved efficiency in gene delivery and expression compared to existing AAV vectors. AAV capsid engineering has been employed to develop AAV vectors that meet these requirements.

Directed evolution is a high-throughput screening method widely employed for engineering improved biomolecules. Directed evolution mimics the process of natural selection through repeated cycles of genetic variation and selection. Directed evolution of AAV capsids involves introducing mutations into the wild-type AAV capsid genes to generate libraries of AAV capsids with diverse sequences, from which variants exhibiting desired properties are screened to identify novel capsid variants.

Recombinant AAV vectors with high tissue specificity and improved gene delivery efficiency have been developed and utilized through AAV capsid engineering (Korean Patent No. 2234930). In particular, hereditary retinal diseases are often single-gene disorders, making them promising targets for gene therapy. Several AAV variants, such as AAV2-7m8, AAV2.GL, and AAV2.NN, have been developed for this purpose. However, there remains a need for AAV variants with superior transduction capabilities that can deliver genes with high specificity to target retinal cells and efficiently induce gene expression.

In this context, the inventors of the present invention have screened an AAV2 variant from an AAV library generated through directed evolution, using human retinal organoids, which exhibits enhanced gene delivery efficacy to retinal cells, thereby completing the present disclosure.

### SUMMARY

An object of the present disclosure is to provide an AAV2 variant capsid protein exhibiting enhanced gene delivery efficiency to retinal cells.

Another objective is to provide a recombinant AAV2 vector comprising an AAV2 variant capsid protein exhibiting enhanced gene delivery efficiency to retinal cells.

Yet another objective is to provide a use of the recombinant AAV2 vector comprising an AAV2 variant capsid protein exhibiting enhanced gene delivery efficiency to retinal cells for the treatment of ocular diseases.

Additional aspects will be set forth in part in the description which follows and, in part, will be apparent from the description, or may be learned by practice of the presented embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows a schematic diagram of a directed evolution method for engineering AAV2 variant capsid proteins according to an embodiment of the present disclosure. A capsid gene (cap) encoding a wild-type AAV2 capsid protein was subjected to error-prone PCR to generate cap gene variants, which were then packaged to construct an AAV library, and the library was transduced into human retinal organoids to select AAVs having tropism for retinal cells. Thereafter, tdTomato+photoreceptor cells were selected from human retinal organoids, from which the cap genes were obtained, analyzed, and packaged to construct a photoreceptor tropic AAV library, and by repeating this process of transduction, selection, and analysis, AAV2 variant capsid proteins were selected that exhibited improved transduction into retinal cells compared to AAVs having wild-type AAV2 capsids.

FIG. 2 illustrates the three-dimensional structure of the RO3 variant according to an embodiment of the present disclosure, in which the positions of point mutations relative to the wild-type AAV2 capsid protein are indicated, and shows, on the right, enlarged views of the outer and inner surfaces of the regions having the point mutations.

FIG. 3 illustrates the structure of a recombinant AAV2 variant vector comprising the RO3 variant according to an embodiment of the present disclosure.

FIG. 4 shows the expression patterns of a target gene observed after subretinal administration of recombinant AAV2 vectors carrying a gene encoding green fluorescent protein (GFP) as the target gene, comprising either the RO3 variant according to an embodiment of the present disclosure or wild-type AAV2 (RO3-GFP vs. AAV2-GFP).

FIG. 5 shows the expression patterns of a target gene observed after intravitreal administration of recombinant AAV2 vectors carrying a gene encoding green fluorescent protein (GFP) as the target gene, comprising either the RO3 variant according to an embodiment of the present disclosure or wild-type AAV2 (RO3-GFP vs. AAV2-GFP). In the Figure, RGC refers to a retinal ganglion cell, INL refers to an inner nuclear layer, ONL refers to an outer membrane layer, PR refers to a photoreceptor cell, and RPE refers to a retinal pigment epithelium cell.

FIGS. 6A and 6B show the expression of GFP observed by immunostaining after transducing human retinal organoids with recombinant AAV2 vectors carrying GFP as a target gene, comprising either the RO3 variant according to an embodiment of the present disclosure or wild-type AAV2 (RO3-GFP vs. AAV2-GFP). FIG. 6A shows yellow regions resulting from the merge of tdTomato, a photoreceptor-specific fluorescent protein, and GFP, and FIG. 6B shows differences in GFP gene expression in human retinal organoids.

FIG. 7 shows the results of examining expression in Muller glial cells and photoreceptor cells by immunostaining after transducing human retinal organoids with recombinant AAV2 vectors carrying GFP as a target gene, comprising either the RO3 variant according to an embodiment of the present disclosure or wild-type AAV2 (RO3-GFP vs. AAV2-GFP).

FIG. 8 shows the results observed after transducing human retinal organoids with recombinant AAV2 vectors carrying GFP as a target gene, comprising either the RO3 variant according to an embodiment of the present disclosure or wild-type AAV2 (RO3-GFP vs. AAV2-GFP), including (a) FACS (fluorescence-activated cell sorting) analysis of tdTomato and GFP fluorescence, (b) comparison of viral genome copy numbers of AAV2 and RO3 in photoreceptor cells, and (c) comparison of mRNA expression of AAV2 and RO3 in photoreceptor cells.

### DETAILED DESCRIPTION

Reference will now be made in detail to embodiments, examples of which are illustrated in the accompanying drawings, wherein like reference numerals refer to like elements throughout. In this regard, the embodiments may have different forms and should not be construed as being limited to the descriptions set forth herein. Accordingly, the embodiments are merely described below, by referring to the figures, to explain aspects of the description. As used herein, the term “and/or” includes any and all combinations of one or more of the associated listed items. Expressions such as “at least one of,” when preceding a list of elements, modify the entire list of elements and do not modify the individual elements of the list.

One aspect of the present disclosure provides an AAV2 variant capsid protein comprising point mutations H38P, 1698V, N705A, V708A, T716N, V719L, S721T, and N734P at positions corresponding to positions 38, 698, 705, 708, 716, 719, 721, and 734 of the wild-type adeno-associated virus (AAV) 2 variant capsid protein of SEQ ID NO: 1.

The AAV2 variant capsid protein of the present disclosure was obtained by engineering the wild-type AAV2 capsid protein through directed evolution to select AAVs with tropism for retinal cells, specifically, photoreceptor cells, and characterizing their structure and gene delivery activity.

“AAV” is an abbreviation for adeno-associated virus and refers to the virus itself or derivatives thereof. Unless otherwise specified, AAV includes all of its subtypes and naturally occurring and recombinant forms. AAV is a non-pathogenic parvovirus consisting of a 4.7 kb long single-stranded DNA genome enclosed within a non-enveloped icosahedral capsid. The genome includes three open reading frames (ORFs) flanked by inverted terminal repeats (ITRs) that function as the viral origins of replication and signals for packaging: the rep ORF encodes four non-structural proteins that play roles in viral replication, transcriptional regulation, site-specific integration, and virion assembly; the cap ORF encodes three structural proteins (VP1-3) that assemble to form the 60-mer viral capsid; and an ORF present as an alternative reading frame within the cap gene encodes the assembly-activating protein (AAP), a viral protein that localizes AAV capsid proteins to the nucleolus and functions in capsid assembly.

The genome sequences of various AAV serotypes, the sequences of their natural terminal repeats (TRs), the Rep proteins, and capsid subunits are known in the art. These sequences can be found in the literature or in public databases such as GenBank. For example, the genome sequence of AAV2 is deposited in GenBank under Accession No. NC_001401.

As used herein, the term “tropism” refers to the preferential targeting of cells of a particular host species or of a particular cell type therein by a virus (for example, AAV), and is used interchangeably with “taxis.” For example, a virus that can infect retinal cells with high specificity has increased tropism for retinal cells compared to non-retinal cells. Because the capsid proteins of AAV viruses determine infectivity to target cells or tissues, tropism or taxis is used as a selection pressure in capsid engineering through directed evolution to develop AAV variants capable of delivering genes to target cells or tissues with high specificity and efficiency.

As used herein, the term “retinal cell” refers to all types of cells present in the retina, for example, retinal ganglion cells, bipolar cells, photoreceptors, Muller glia cells, horizontal cells, amacrine cells, astrocytes, microglia, retinal vascular cells, and retinal pigment epithelial cells, but is not limited thereto.

As used herein, the term “gene delivery efficacy” refers to the ability or activity of an AAV vector to specifically deliver and express a gene in target cells or tissues, and is used interchangeably herein with “infectivity” or “transduction efficacy.”

The AAV2 variant capsid protein of the present disclosure comprises point mutations H38P, 1698V, N705A, V708A, T716N, V719L, S721T, and N734P compared to the wild-type AAV2 capsid protein, and has been confirmed to deliver a target gene with high efficiency by transducing retinal pigment epithelial cells, photoreceptor cells, ganglion cells, Muller glial cells, and bipolar cells via intravitreal injection compared to the wild-type AAV2, and is designated as the “RO3” variant.

The RO3 variant according to the present disclosure has been confirmed, upon intravitreal injection, to pass through the inner limiting membrane (ILM) and deliver a target gene to photoreceptor cells and retinal pigment epithelial cells, inducing strong gene expression, and to traverse the retinal layers, resulting in delivery to deeper retinal cells and increased expression of the target gene compared to wild-type AAV2. Furthermore, compared to wild-type AAV2, enhanced gene expression and photoreceptor cell-specific gene delivery have been confirmed in human retinal organoids, demonstrating superior gene delivery efficiency.

The wild-type AAV2 capsid protein comprises the amino acid sequence of SEQ ID NO: 1. The RO3 variant according to the present disclosure comprises point mutations H38P, 1698V, N705A, V708A, T716N, V719L, S721T, and N734P at positions corresponding to residues 38, 698, 705, 708, 716, 719, 721, and 734 of the amino acid sequence of SEQ ID NO: 1.

In an embodiment of the present disclosure, the AAV2 variant capsid protein may comprise SEQ ID NO: 2 or an amino acid sequence having at least 85%, at least 90%, or at least 95% sequence identity thereto.

In an embodiment of the present disclosure, the AAV2 variant capsid protein may consist of SEQ ID NO: 2 or an amino acid sequence having at least 85%, at least 90%, or at least 95% sequence identity thereto.

As used herein, the term “sequence identity” refers to the percentage of amino acids or nucleotides that are the same at corresponding positions between compared polypeptides or polynucleotides, and may be used interchangeably with “homology” with respect to a sequence. For example, the percent homology (%) between two polynucleotide sequences is calculated by optimally aligning the two sequences for comparison and comparing the nucleotides at corresponding nucleotide positions, wherein a nucleotide at a given position in the first sequence is considered identical to the nucleotide at the corresponding position in the second sequence, if they are the same. Sequence homology can be determined using software such as BLASTP, BLASTN, or FASTA.

In an embodiment of the present disclosure, the AAV2 variant capsid protein may confer increased infectivity to retinal cells compared to the wild-type AAV2 capsid protein.

In an embodiment of the present disclosure, the retinal cells may be selected from the group consisting of retinal ganglion cells, bipolar cells, photoreceptor cells, Muller glial cells, horizontal cells, amacrine cells, astrocytes, microglia, retinal vascular cells, and retinal pigment epithelial cells.

Another aspect of the present disclosure provides an isolated nucleic acid encoding an AAV variant capsid protein comprising point mutations H38P, 1698V, N705A, V708A, T716N, V719L, S721T, and N734P at positions corresponding to residues 38, 698, 705, 708, 716, 719, 721, and 734 of the wild-type AAV2 capsid protein of SEQ ID NO: 1.

In an embodiment of the present disclosure, the isolated nucleic acid may comprise a nucleotide sequence of SEQ ID NO: 4 or a nucleotide sequence having at least 95% sequence homology thereto.

Another aspect of the present disclosure provides a host cell comprising an isolated nucleic acid encoding an AAV variant capsid protein comprising point mutations H38P, 1698V, N705A, V708A, T716N, V719L, S721T, and N734P at positions corresponding to residues 38, 698, 705, 708, 716, 719, 721, and 734 of the wild-type AAV2 capsid protein of SEQ ID NO: 1.

The host cells may be used to generate recombinant AAV virions comprising the AAV variant capsid proteins. In this case, the host cell is referred to as a “packaging cell.” The host cell may be selected from a variety of cells, including but not limited to 293 cells, COS cells, Hela cells, CHO cells, HEK cells, and AAV293 cells.

In an embodiment of the present disclosure, the isolated nucleic acid is introduced stably or transiently into a host cell using methods including but not limited to, electroporation, calcium phosphate precipitation, and liposome-mediated transfection.

Another aspect of the present disclosure provides a recombinant AAV vector comprising an AAV2 variant capsid protein comprising point mutations H38P, 1698V, N705A, V708A, T716N, V719L, S721T, and N734P at positions corresponding to residues 38, 698, 705, 708, 716, 719, 721, and 734 of the wild-type AAV2 capsid protein of SEQ ID NO: 1, and a heterologous nucleic acid comprising a sequence encoding a target product.

As used herein, the term “recombinant AAV vector” refers to an AAV vector comprising a polynucleotide sequence that is not derived from AAV, i.e., a polynucleotide sequence heterologous to AAV, and is used interchangeably with “rAAV.” Generally, in a recombinant AAV vector, the heterologous polynucleotide sequence is a nucleic acid sequence encoding a target product to be delivered to a target cell.

As used herein, the term “heterologous” refers to a nucleic acid originating from a genetically distinct source from the AAV2 vector, for example, a different species.

In an embodiment of the present disclosure, the recombinant AAV vector may exhibit enhanced transduction of retinal tissues/cells compared to an AAV vector comprising a wild-type AAV2 capsid protein (AAV2 vector).

A recombinant AAV vector according to an embodiment of the present disclosure may be used to prepare recombinant AAV virions. When introduced into a suitable host cell, the recombinant AAV vector may provide for the generation of the corresponding recombinant AAV virions.

In an embodiment of the present disclosure, the target product may be a polypeptide or a nucleic acid. The target product refers to a product that can compensate for or enhance a function that is deficient or lacking in the subject, or reduce or eliminate a defective function, thereby providing a therapeutic effect for a target disease. For example, the target product may be a gene product directly or indirectly associated with an ocular disease such as a retinal disorder, or modulate its activity, or enhance the function of retinal cells. Examples include pigment epithelium-derived factor (PEDF) or rhodopsin.

In an embodiment of the present disclosure, the target product may be a therapeutic nucleic acid, which may target any gene associated with an ocular disease, such as achromatopsia, macular degeneration including wet or dry age-related macular degeneration (AMD), cataracts, pan-choroidal atrophy, glaucoma, optic neuropathy, Marfan syndrome, myopia, nodular choroidal vasculopathy, retinitis pigmentosa, Stargardt disease, Usher syndrome, Leber congenital amaurosis, Leber hereditary optic neuropathy, uveal melanoma, or X-linked retinoschisis or any hereditary disease affecting ocular tissues. The target gene may, for example, be a gene encoding a protein selected from the group consisting of CNGB3, NOS2A, CFH, CF, C2, C3, CFB, HTRA1/LOC, MMP-9, TIMP-3, SLC16A8, GEMIN4, CYP51A1, RIC1, TAPT1, TAFIA, WDR87, APE1, MIP, Cx50, GJA3, GJA8, CRYAA, CRYBB2, PRX, POLR3B, XRCC1, ZNF350, EPHA2, REP1, CALM2, MPP-7, Optineurin, LOX1, CYP1B1, CAV½, MYOC, PITX2, FOXCI, PAX6, LTBP2, Complex I, ND, OPA1, RPE65, FBN1, TGFBR2, MTHFR, MTR, MTRR, HGF, C-MET, UMODL1, MMP-½, CBS, IGF-1, UHRF1BP1L, PTPRR, PPFIA2, P4HA2, SERPING1, PEDF, ARMS2-HTRA1, FGD6, ABCG1, LOC387715, CETP, NRL, RDH12, PRPH2 (RDS), RHO, RPGR, SNRNP200, NR2E3, IMPDH1, CRX, HK1, IMPDH2, PRPF3, AGBL5, ABCA1, ABCA4, CRB1, USH2A, NRP1, ND4, RLBP1, PTEN, BAP1, GNAQ, GNA11, DDEF1, SF3B1, EIF1AX, CDKN2A, p14ARF, HERC2/OCA2, VEGF, and RSI, but is not limited thereto.

In an embodiment of the present disclosure, the target product may be a therapeutic protein or polypeptide, and may for example, be selected from the group consisting of CNGB3, NOS2A, CFH, CF, C2, C3, CFB, HTRA1/LOC, MMP-9, TIMP-3, SLC16A8, GEMIN4, CYP51A1, RIC1, TAPT1, TAFIA, WDR87, APE1, MIP, Cx50, GJA3, GJA8, CRYAA, CRYBB2, PRX, POLR3B, XRCC1, ZNF350, EPHA2, REP1, CALM2, MPP-7, Optineurin, LOX1, CYP1B1, CAV½, MYOC, PITX2, FOXCI, PAX6, LTBP2, Complex I, ND, OPA1, RPE65, FBN1, TGFBR2, MTHFR, MTR, MTRR, HGF, C-MET, UMODL1, MMP-½, CBS, IGF-1, UHRF1BP1L, PTPRR, PPFIA2, P4HA2, SERPING1, PEDF, ARMS2-HTRA1, FGD6, ABCG1, LOC387715, CETP, NRL, RDH12, PRPH2 (RDS), RHO, RPGR, SNRNP200, NR2E3, IMPDH1, CRX, HK1, IMPDH2, PRPF3, AGBL5, ABCA1, ABCA4, CRB1, USH2A, NRP1, ND4, RLBP1, PTEN, BAP1, GNAQ, GNA11, DDEF1, SF3B1, EIF1AX, CDKN2A, p14ARF, HERC2/OCA2, VEGF, and RSI, but is not limited thereto.

Another aspect of the present disclosure provides the use of the aforementioned recombinant AAV vector in the treatment of ocular diseases.

In an embodiment of the present disclosure, a pharmaceutical composition for treating an ocular disease comprising the aforementioned recombinant AAV vector is provided.

In an embodiment of the present disclosure, a method of treating an ocular disease in a subject in need thereof, comprising is provided, comprising administering a therapeutically effective amount of the recombinant AAV vector described above to the subject.

As used herein, the term “ocular disease” refers to a disease of the eye or a specific part or region thereof, for example, a disease related to the retina or choroid.

In an embodiment of the present disclosure, the ocular disease may be a retinal disease, a choroidal disease, or a retinal/choroidal disease.

In an embodiment of the present disclosure, the ocular disease may be selected from retinal vascular disease, optic neuropathy, hereditary retinal disease, hereditary choroidal disease, and intraocular tumor.

In an embodiment of the present disclosure, the ocular disease may be selected from retina-vitreous-choroid related diseases (retinal, vitreous, and choroidal disorders), retinal vascular diseases, macular diseases, hereditary retinal diseases, hereditary vitreous diseases, hereditary choroidal diseases, intraocular inflammatory diseases, intraocular tumors, glaucoma, and optic neuropathy.

In an embodiment of the present disclosure, the ocular disease may be a retinal vascular disease including age-related macular degeneration, diabetic retinopathy, retinal vascular occlusion such as retinal artery/vein occlusion, and macular telangiectasia; an optic neuropathy including glaucoma; a hereditary retinal/choroidal disease including uveitis, Leber congenital amaurosis, retinitis pigmentosa, Usher syndrome, Stargardt disease, Best disease, X-linked retinoschisis, congenital stationary night blindness, choroideremia, achromatopsia, cone dystrophy, gyrate atrophy, and Bardet-Biedl syndrome; or an intraocular tumor such as uveal melanoma, intraocular lymphoma, or retinoblastoma, but is not limited thereto.

In an embodiment of the present disclosure, the ocular disease may be selected from macular degeneration, diabetic retinopathy, retinal vascular occlusion, macular telangiectasia, retinopathy of prematurity, myopic degeneration, retinitis pigmentosa, Leber congenital amaurosis, Best disease, Stargardt disease, congenital stationary night blindness, X-linked retinoschisis, Bietti crystalline dystrophy, total achromatopsia, cone dystrophy, cone-rod dystrophy, maculopathy, Usher syndrome, Bardet-Biedl syndrome and other syndromic retinitis pigmentosa, pan choroidal atrophy, central serous chorioretinopathy (CSR), gyrate atrophy of choroid and retina, glaucoma, other optic neuropathies, uveitis, uveal melanoma, intraocular lymphoma, retinoblastoma, retinal detachment, and other retinal injury.

In an embodiment of the present disclosure, the recombinant AAV vector may be administered via ocular injection by intravitreal injection, subretinal injection, or suprachoroidal injection, or via any suitable route for delivery to the retina, including, but not limited to, periocular, intravenous, intra-arterial, and intranasal administration.

In an embodiment of the present disclosure, the recombinant AAV vector may be administered via intravitreal injection. The recombinant AAV vector of the present disclosure, when administered via intravitreal injection, can move through the vitreous, pass through the internal limiting membrane (ILM), and traverse the retinal layers more efficiently compared to an AAV vector comprising the wild-type AAV2 capsid protein.

In an embodiment of the present disclosure, the recombinant AAV vector may be administered via intraocular injection.

The recombinant AAV vector according to the present disclosure, when administered via intravitreal injection, has an increased ability to traverse the inner limiting membrane (ILM) and deliver and express a heterologous nucleic acid in retinal cells compared to AAV2 vector comprising a wild-type capsid protein.

The recombinant AAV vector according to the present disclosure can traverse the ILM and further pass through cell layers including Muller glial cells and bipolar cells, to reach photoreceptor cells or retinal pigment epithelium (RPE) cells. For example, when administered via intravitreal injection, the recombinant AAV vector according to the present disclosure can traverse the ILM and further pass through cell layers including Muller glial cells and bipolar cells, to reach photoreceptor cells and/or RPE cells.

In some embodiments, the recombinant AAV vector selectively infects retinal cells and, for example, can infect retinal cells with 10-fold, 15-fold, 20-fold, 25-fold, 50-fold, or greater specificity compared to non-retinal cells such as cells outside the eye.

As used herein, the term “therapeutically effective amount” refers to an amount sufficient to alleviate (for example, relieve, reduce, diminish) at least one of the symptoms associated with a disease condition. For example, a therapeutically effective amount for in vivo injection, i.e., direct injection into the retina, may be in the range of about 10⁶ to about 10¹⁵, for example, from about 10⁸ to about 10¹² rAAV virions. For in vitro transduction, an effective amount of rAAV virions delivered to cells may be in the range of about 10⁸ to about 10¹³ rAAV virions. The therapeutically effective amount of the recombinant AAV vector according to the present disclosure may be readily determined by one of ordinary skill in the art by taking into account factors such as the target disease, the age, sex, body weight of the subject, and the severity of the disease to be treated.

In an embodiment of the present disclosure, the recombinant viral vector is administered in an amount sufficient to achieve infection (or transduction) and expression of the heterologous nucleic acid sequence in retinal cells of the subject.

In an embodiment of the present disclosure, the retinal cells may be selected from the group consisting of retinal ganglion cells, bipolar cells, photoreceptor cells, Muller glial cells, horizontal cells, amacrine cells, astrocytes, microglia, retinal vascular cells, and retinal pigment epithelium (RPE) cells.

In an embodiment of the present disclosure, the retinal cells are photoreceptor cells, for example, cone cells and/or rod cells.

In an embodiment of the present disclosure, the retinal cells are retinal ganglion cells (RGCs).

In an embodiment of the present disclosure, the retinal cells are RPE cells.

In an embodiment of the present disclosure, the pharmaceutical composition may be administered one or more times over various intervals, for example, daily, weekly, monthly, or yearly, to achieve a desired level of gene expression.

In an embodiment of the present disclosure, the pharmaceutical composition may comprise a pharmaceutically acceptable carrier, excipient or additive.

Another aspect of the present disclosure provides an in vitro method for delivering a heterologous nucleic acid encoding a target product into retinal cells, comprising contacting the retinal cells with the above-described recombinant AAV vector.

According to the present disclosure, delivery of heterologous nucleic acids into retinal cells may be used for the treatment of retinal diseases.

In an embodiment, the target product and retinal cells are as described above.

The RO3 variant according to the present disclosure has been confirmed to pass through the ILM upon intravitreal injection, deliver a target gene to retinal cells, including photoreceptor cells and retinal pigment epithelial cells, induce strong gene expression, provide increased gene expression and lateral spreading compared to wild-type AAV2, and exhibit superior gene delivery efficiency through increased photoreceptor cell-specific gene delivery and gene expression in human retinal organoids compared to wild-type AAV2. Therefore, the RO3 variant according to the present disclosure can be usefully employed in gene therapy for the treatment of ocular diseases.

One or more embodiments will be described in detail with reference to the following examples. However, these examples are for illustrative purposes only and are not intended to limit the scope of one or more embodiments.

### Example 1. Screening of RO3 Variants

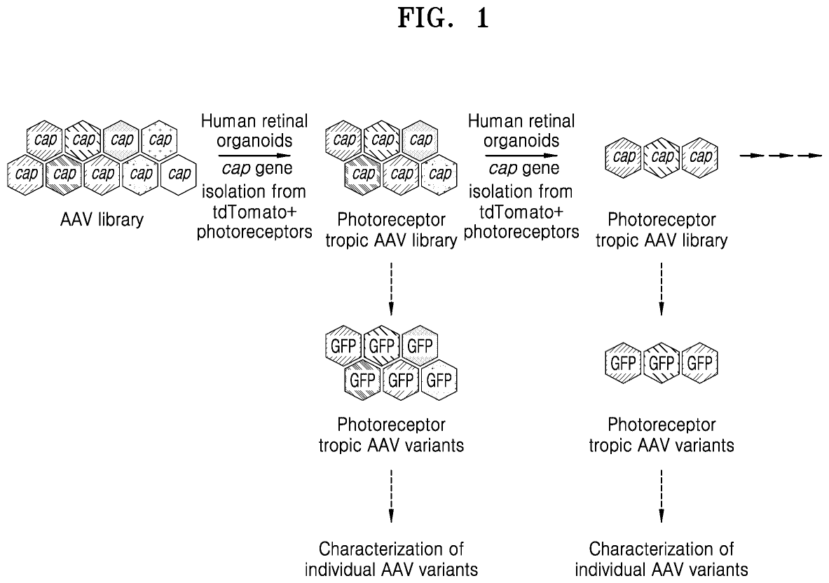

AAV2 capsid engineering was performed through directed evolution to identify AAV2 capsid variants capable of efficiently delivering and expressing a target gene in retinal cells. FIG. 1 illustrates a schematic diagram of the directed evolution method.

In vitro and in vivo directed evolution technologies have been utilized to develop AAV variants with improved properties compared to conventional AAV-based gene delivery vectors. Directed evolution technologies are known in the art and are described, for example, in PCT Publication No. WO 2014/194132. Directed evolution is a capsid engineering method that mimics natural evolution through iterative genetic diversification and selection processes, enabling the accumulation of beneficial mutations that gradually improve the function of biomolecules such as AAV-based virions. In this approach, various mutations are introduced into the wild-type AAV capsid (cap) gene by using techniques such as error-prone PCR to introduce random point mutations, DNA shuffling to generate random chimeras, random peptide insertions, or transposon-mediated mutagenesis. The mutated AAV cap genes are packaged into AAV particles to generate a library of AAV variants, and selective pressure is applied to isolate unique variants with superior phenotypes capable of overcoming gene delivery barriers.

Specifically, a pool of plasmids was generated by introducing random point mutations into the wild-type AAV2 cap genes using error-prone PCR. 7-70 ng of AAV plasmid library, 25 μg of pBluescript, and 25 μg of pHelper were complexed using calcium phosphate and transfected into AAV-293 cells (#240073, Agilent Technologies) to perform AAV packaging, thereby producing a pooled AAV library carrying the cap gene information of each variant.

In addition, human retinal organoids used to evaluate the gene delivery efficacy of AAV2 variants to retinal cells were prepared as described in Wahlin, K. J. et al. Photoreceptor Outer Segment-like Structures in Long-Term 3D Retinas from Human Pluripotent Stem Cells. Sci Rep 7, 766 (2017). Specifically, the human retinal organoids were prepared using human embryonic stem cells that express the tdTomato fluorescent protein in a photoreceptor-specific manner. Human embryonic stem cells expressing tdTomato fluorescent protein in a photoreceptor cell-specific manner were cultured on Matrigel (Corning, NY, USA)-coated plates using mTesR1 basal medium (Stem Cell Technologies, Vancouver, Canada). Before inducing differentiation into retinal organoids, the cells were cultured for 4-5 days. The cultured cells were then dissociated using Accutase (BD Biosciences, CA, USA) and aggregated at a density of 3,000 cells per well in U-bottom, low-cell-adhesion 96-well plates (Corning). Thereafter, the cells were differentiated and cultured for at least 30 weeks to obtain human retinal organoids. All cultures for the preparation of human retinal organoids were maintained at 37° C. in a humidified environment with 5% carbon dioxide.

1×10¹⁰ vg of an AAV library pool was transduced into the obtained human retinal organoids, and two weeks later, the organoids were collected, lysed, and DNA was extracted using a DNA mini kit (Qiagen). The cap genes of AAV variants exhibiting photoreceptor tropism in the retinal organoids were amplified using AAV cap gene-specific primers (forward primer: 5′-GCGGAAGCTTCGATCAACTACG-3′, and reverse primer: 5′-CGCAGAGACCAAAGTTCAACTGA′-3′).

A photoreceptor tropic AAV library was generated by packaging the cap genes of selected AAV variants, and the library was transduced into human retinal organoids. tdTomato+photoreceptor cells containing AAV were then sorted by FACS, the cap genes were recovered, and this process was iteratively repeated to isolate photoreceptor tropic AAV variants.

AAV variants that were confirmed to exhibit photoreceptor tropism in human retinal organoids were screened to isolate a single variant exhibiting both (i) enhanced gene delivery into photoreceptor cells following intravitreal injection in C57BL/6 mice, and (ii) improved photoreceptor-targeted gene expression in human retinal organoids.

Sequence analysis of this variant revealed that it harbors point mutations H38P, 1698V, N705A, V708A, T716N, V719L, S721T, and N734P compared to wild-type AAV2, and this variant was designated as the “RO3” variant.

The sequences of the wild-type AAV2 capsid protein (SEQ ID NO: 1) and the RO3 capsid protein (SEQ ID NO: 2) in the RO3 region containing point mutations are compared below:

Wild-type AAV2 (1-50)
MAADGYLPDW LEDTLSEGIR QWWKLKPGPP PPKPAERHKD
DSRGLVLPGY
RO3 (1-50)
MAADGYLPDW LEDTLSEGIR QWWKLKPGPP PPKPAERPKD
DSRGLVLPGY
Wild-type AAV2 (651-700)
TPVPANPSTT FSAAKFASFI TQYSTGQVSV EIEWELQKEN
SKRWNPEIQY
RO3 (651-700)
TPVPANPSTT FSAAKFASFI TQYSTGQVSV EIEWELOKEN
SKRWNPEVQY
Wild-type AAV2 (701-735)
TSNYNKSVNV DFTVDTNGVY SEPRPIGTRY LTRNL
RO3 (701-735)
TSNYAKSANV DFTVDNNGLY TEPRPIGTRY LTRPL

The numbers in parentheses indicate the positions of amino acids in each sequence, and mutations compared to the wild-type AAV2 capsid are shown in bold.

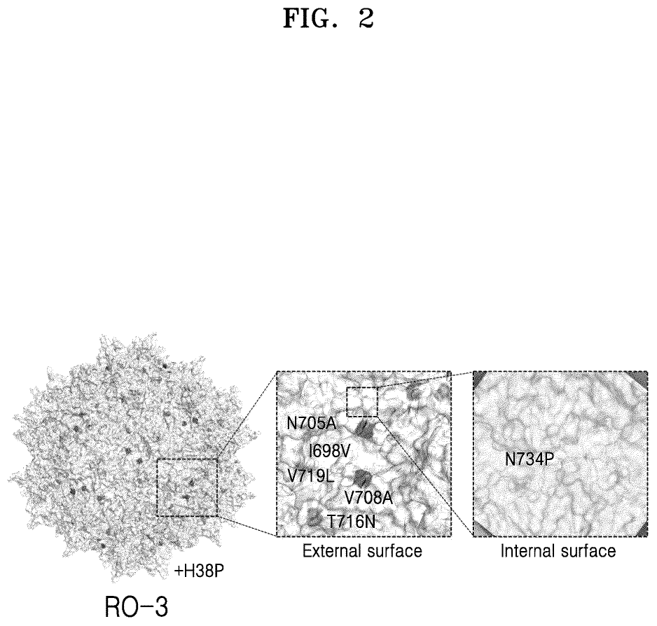

FIG. 2 illustrates the three-dimensional structure of the RO3 variant, with the locations of point mutations on the external and internal surfaces indicated.

The RO3 variant, compared to the wild-type AAV2 capsid protein, harbors point mutations H38P, 1698V, N705A, V708A, T716N, V719L, S721T, and N734P, and exhibits photoreceptor specificity, as well as increased spread to photoreceptors, resulting in enhanced transduction.

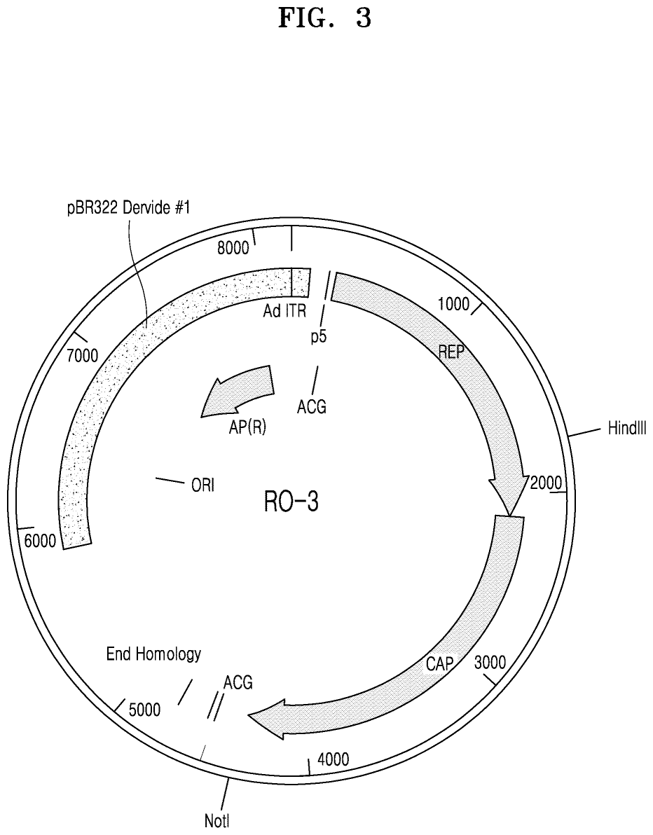

FIG. 3 illustrates the structure of a vector comprising the RO3 variant.

### Example 2. Gene Delivery Using RO3 Variants

The gene delivery properties of the RO3 variant, which was selected in Example 1 for improved specificity and gene delivery efficiency to retinal cells compared to wild-type AAV2, were analyzed.

### (1) Subretinal Injection

A recombinant vector harboring a gene encoding green fluorescent protein (GFP) as the target gene and the RO3 variant (rAAV-GFP) was injected subretinally into mice, and gene expression in photoreceptor cells was observed.

Specifically, recombinant AAV2 vectors containing either wild-type AAV2 or RO3 capsid, each carrying a gene encoding GFP were subretinally injected into 8-week-old C57BL/6 mice at a dose of 1.4×10¹⁰ vg/μL (2 μL). Four weeks later, the mice were sacrificed, and the eyes were collected. The collected eyes were fixed in 1% paraformaldehyde for 1 hour. After fixation, they were sequentially incubated in 10%, 20%, and 30% sucrose solutions for 1 hour each. For sectioning, the eyes were positioned in OCT (Optimal Cutting Temperature) compound so that the optic nerve and the central corneal junction were aligned horizontally, and rapidly frozen using liquid nitrogen vapor. The eyes were then sectioned at a thickness of 7 μm, and the sections were analyzed for gene expression patterns using a primary antibody against L/M opsin to label cone photoreceptors, a primary antibody against Rhodopsin to label rods, and fluorescently labeled secondary antibodies.

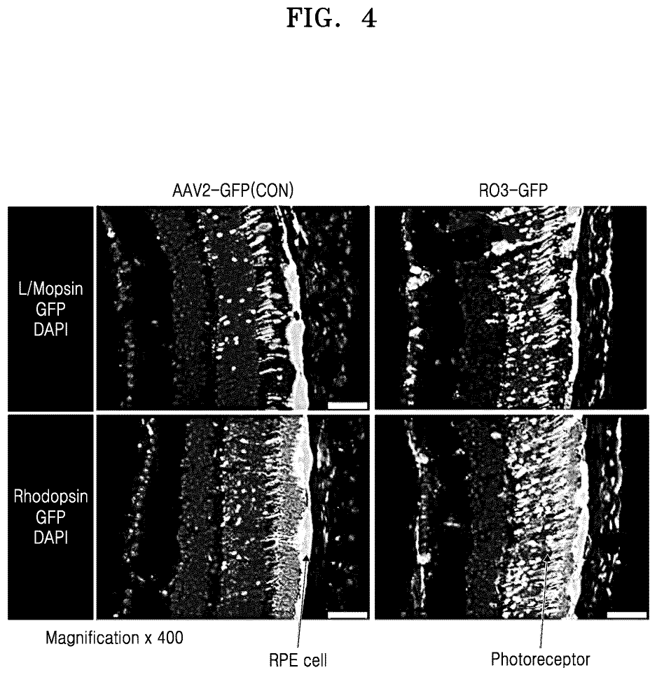

The results are shown in FIG. 4. In the case of wild-type AAV (AAV-GFP), gene expression was observed in the RPE near the injection site, whereas RO3 penetrated deeper from the injection site, and gene expression was detected in photoreceptor cells.

### (2) Intravitreal Injection

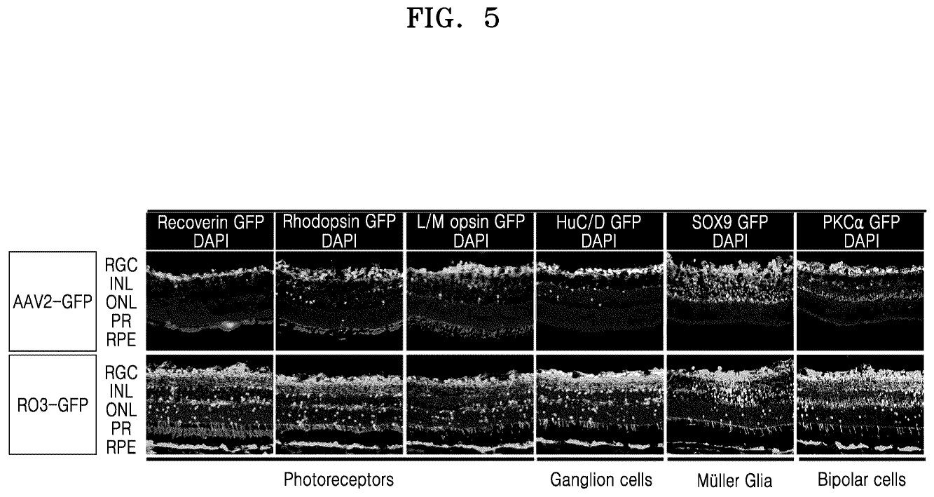

Wild-type AAV2 or RO3 vectors, each harboring a gene encoding GFP as the target gene, were intravitreally injected into 8-week-old C57BL/6 mice at a dose of 2×10¹⁰ vg/μL (2 μL). Four weeks later, the mice were sacrificed, and the eyes were collected. The collected eyes were fixed in 1% paraformaldehyde for 1 hour. After fixation, they were sequentially incubated in 10%, 20%, and 30% sucrose solutions for 1 hour each. For sectioning, the eyes were positioned in OCT (Optimal Cutting Temperature) compound so that the optic nerve and the central corneal junction were aligned horizontally, and rapidly frozen using liquid nitrogen vapor. The eyes were then sectioned at a thickness of 7 μm to prepare sections, and immunofluorescence staining was performed using retinal cell-specific primary antibodies and fluorescently labeled secondary antibodies to examine GFP expression. The following antibodies were used to label retinal cells:
Recoverin antibody: used to label photoreceptor cells
L/M opsin antibody: used to label cone photoreceptor cells
Rhodopsin antibody: used to label rod photoreceptor cells
HuC/D antibody: used to label ganglion cells
Sox9 antibody: used to label Muller glia cells
PKCα antibody: used to label bipolar cells
The results are shown in FIG. 5.

The RO3 variant (RO3-GFP) was found to transduce retinal pigment epithelial cells, photoreceptor cells, ganglion cells, Muller glial cells, and bipolar cells in the mouse retina, delivering the GFP fluorescent gene. In contrast, AAV2 (AAV2-GFP) failed to deliver the GFP gene to retinal pigment epithelial cells and photoreceptor cells, and GFP expression was observed only up to the inner nuclear layer (INL), where Muller glial cell nuclei and bipolar cells are located.

### (3) Human Retinal Organoids

Human retinal organoids expressing tdTomato fluorescence in a photoreceptor-specific manner, prepared as described in Example 1, were transduced with a dose of 5×10¹⁰ vg of wild-type AAV2-GFP or RO3-GFP, each harboring a gene encoding GFP as the target gene, and the delivery and expression of the target gene were observed.

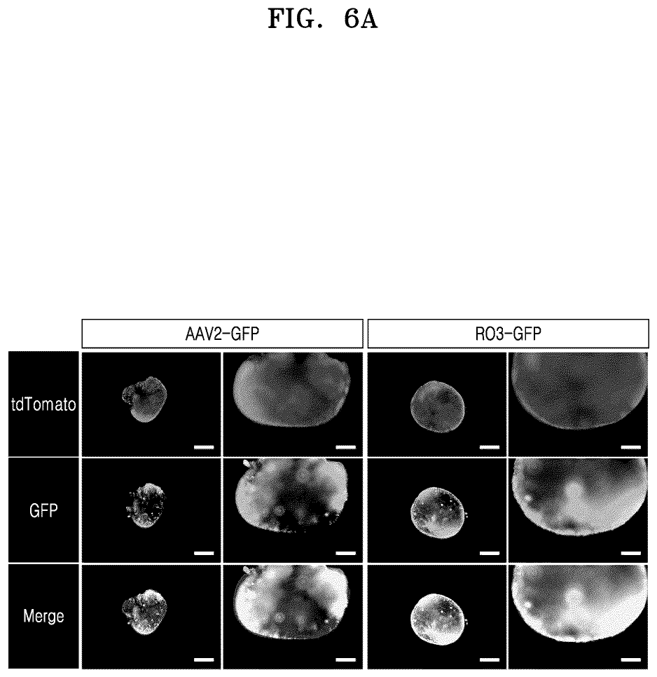

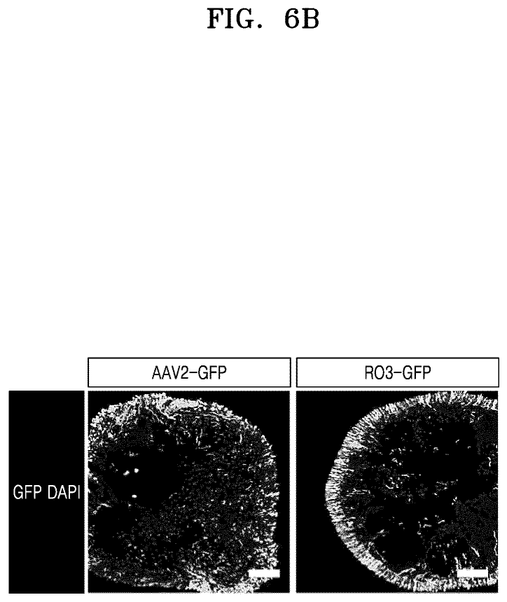

In human retinal organoids, the RO3 variant was found to exhibit higher GFP gene delivery efficiency than AAV2. In addition, in retinal organoids treated with the RO3 variant, areas where photoreceptor-specific tdTomato fluorescence and GFP fluorescence were merged and appeared yellow were broader than those in retinal organoids treated with AAV2. The results are shown in FIG. 6A.

Therefore, the RO3 variant was confirmed to be superior to AAV2 in gene delivery to human photoreceptor cells.

Human retinal organoids transduced with wild-type AAV2-GFP or RO3-GFP were also fixed in a 5% sucrose solution with 4% paraformaldehyde for 25 minutes. After fixation, they were sequentially treated with 10%, 20%, and 30% sucrose solutions for 1 hour each. For sectioning, human retinal organoids were placed in OCT compound and rapidly frozen using liquid nitrogen vapor. The organoids were then sectioned at a thickness of 7 μm to examine differences in GFP gene expression. The results are shown in FIG. 6B. The RO3 variant was confirmed to exhibit higher GFP gene delivery efficiency than AAV2.

Immunofluorescence staining was performed on the prepared sections using retinal cell-labeling primary antibodies and fluorescently labeled secondary antibodies. The antibodies used to label retinal cells were as follows: Recoverin antibody for photoreceptors; L/M opsin antibody for cone photoreceptors; CRALBP antibody and Sox9 antibody for Muller glia cells.

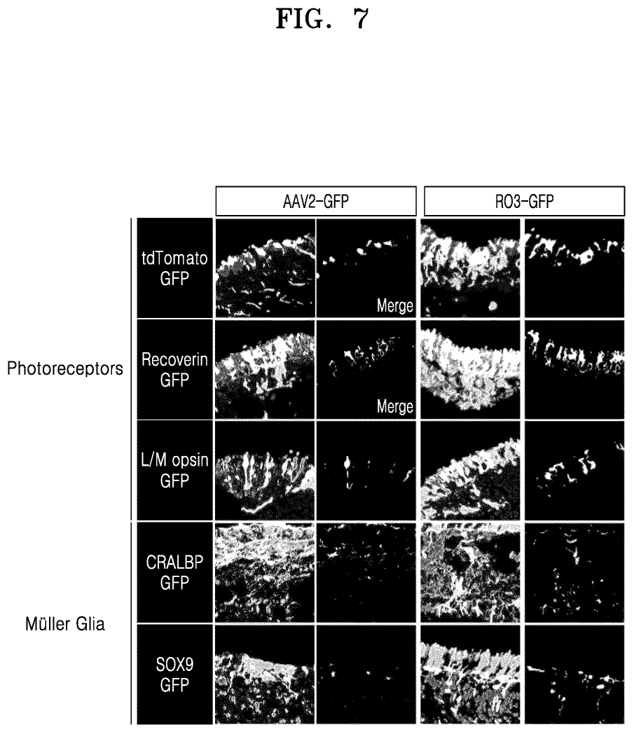

The results of immunostaining are shown in FIG. 7. The expression of the GFP gene delivered by AAV2 and RO3 variant was merged with the fluorescence of the photoreceptor cell-labeling antibody and the fluorescence of the Muller glial cell-labeling antibody, respectively. GFP gene expression mediated by the RO3 variant showed broader areas of colocalization with photoreceptor and Muller glial markers compared to AAV2. Therefore, it was confirmed that the RO3 variant has superior GFP gene delivery efficiency to human photoreceptors and human Muller glial cells relative to AAV2.

### FACS (Fluorescence-Activated Cell Sorting)

Human retinal organoids expressing tdTomato fluorescence in a photoreceptor-specific manner, prepared as described in Example 1, were transduced with a dose of 5×10¹⁰ vg of wild-type AAV2 or RO3, each harboring a gene encoding GFP as the target gene, FACS analysis was performed three weeks later. Human retinal organoids were dissociated using the Papain Dissociation System (Worthington Biochemical Corporation, NJ, USA). FACS was performed using BD FACS Aria II (BD Biosciences, CA, USA), and the resulting data was analyzed with FlowJo software (Tree Star, OR, USA).

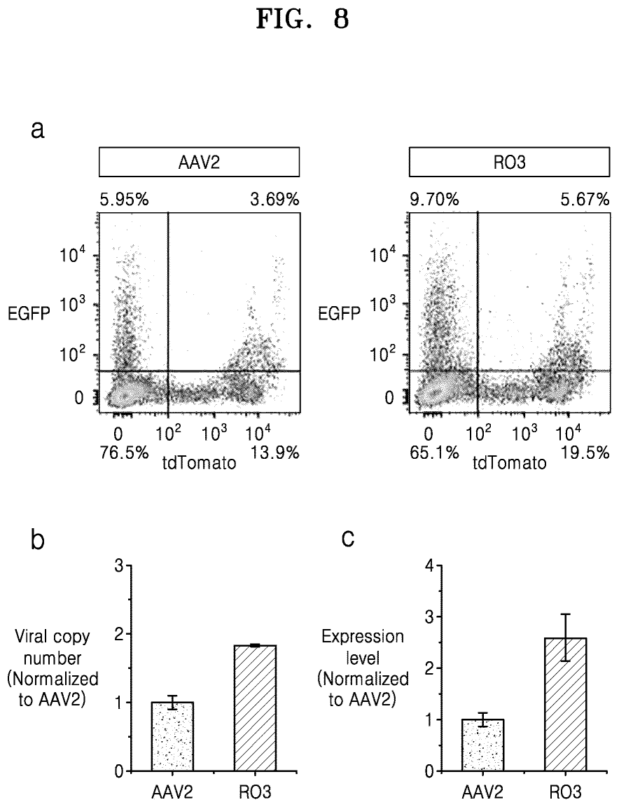

FACS analysis showed that RO3 variant yielded a higher proportion of GFP-positive cells than AAV2 used as a control, with a particularly higher proportion of GFP-positive cells among photoreceptors (tdTomato-positive cells). Additionally, RO3 exhibited higher viral genome copy numbers and GFP mRNA expression in photoreceptors sorted and collected, compared to AAV2. The results are shown in FIG. 8.

These results show that the RO3 variant, selected in Example 1, delivers the target gene to retinal cells more efficiently than wild-type AAV2, crosses the ILM from the injection site to reach photoreceptors, and mediates target gene expression at a higher frequency.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. An adeno-associated virus 2 (AAV2) variant capsid protein comprising point mutations H38P, 1698V, N705A, V708A, T716N, V719L, S721T, and N734P at positions corresponding to positions 38, 698, 705, 708, 716, 719, 721, and 734 of wild-type AAV2 capsid protein of SEQ ID NO: 1.

2. The AAV2 variant capsid protein of claim 1, wherein the AAV2 variant capsid protein comprises the amino acid sequence of SEQ ID NO: 2 or an amino acid sequence having at least 95% sequence identity thereto.

3. The AAV2 variant capsid protein of claim 1, wherein the AAV2 variant capsid protein consists of the amino acid sequence of SEQ ID NO: 2 or an amino acid sequence having at least 95% sequence identity thereto.

4. The AAV2 variant capsid protein of claim 1, wherein the AAV2 variant capsid protein confers increased infectivity to retinal cells compared to a wild-type AAV2 capsid protein.

5. The AAV2 variant capsid protein of claim 4, wherein the retinal cells are selected from the group consisting of retinal ganglion cells, bipolar cells, photoreceptors, Muller glial cells, horizontal cells, amacrine cells, astrocytes, microglial cells, retinal vascular cells, and retinal pigment epithelial cells.

6. An isolated nucleic acid encoding the AAV2 variant capsid protein of claim 1.

7. A host cell comprising an isolated nucleic acid encoding the AAV2 variant capsid protein of claim 1.

8. A recombinant AAV vector comprising: an AAV2 variant capsid protein comprising point mutations H38P, 1698V, N705A, V708A, T716N, V719L, S721T, and N734P at positions corresponding to positions 38, 698, 705, 708, 716, 719, 721, and 734 of wild-type AAV2 capsid protein of SEQ ID NO: 1, and a heterologous nucleic acid comprising a sequence encoding a target product.

9. The recombinant AAV vector of claim 8, wherein the recombinant AAV vector confers increased transduction of retinal cells compared to an AAV vector comprising a wild-type AAV2 capsid protein.

10. The recombinant AAV vector of claim 8, wherein the target product is a polypeptide or nucleic acid.

11. A method for treating an ocular disease in a subject in need thereof, comprising administering the recombinant AAV vector of claim 8.

12. The method of claim 11, wherein the ocular disease is selected from the group consisting of retinal, vitreous, and choroidal disorders, retinal vascular diseases, macular diseases, hereditary retinal diseases, hereditary vitreous diseases, hereditary choroidal diseases, intraocular inflammatory diseases, intraocular tumors, glaucoma, and optic neuropathies.

13. The method of claim 11, wherein the ocular disease is selected from the group consisting of macular degeneration, diabetic retinopathy, retinal vascular occlusion, macular telangiectasia, retinopathy of prematurity, myopic degeneration, retinitis pigmentosa, Leber congenital amaurosis, Best disease, Stargardt disease, congenital stationary night blindness, X-linked retinoschisis, Bietti crystalline dystrophy, total achromatopsia, cone dystrophy, cone-rod dystrophy, maculopathy, Usher syndrome, Bardet-Biedl syndrome, syndromic retinitis pigmentosa, pan choroidal atrophy, central serous chorioretinopathy (CSR), gyrate atrophy of choroid and retina, glaucoma, optic neuropathy, uveitis, uveal melanoma, intraocular lymphoma, retinoblastoma, retinal detachment, and retinal injury.

14. The method of claim 11, wherein the recombinant AAV vector is administered via intravitreal injection, subretinal injection, or suprachoroidal injection.

15. An in vitro method for delivering a heterologous nucleic acid to retinal cells, comprising contacting the recombinant AAV vector of claim 8 with the retinal cells.

16. The method of claim 15, wherein the retinal cells are selected from the group consisting of retinal ganglion cells, bipolar cells, photoreceptors, Muller glial cells, horizontal cells, amacrine cells, astrocytes, microglial cells, retinal vascular cells, and retinal pigment epithelial cells.
